(19) United States
(12) Patent Application Publication
(10) Pub. No.: US 2017/0145346 A1
(43) Pub. Date: May 25, 2017

(54) SYNERGISTIC SURFACTANT COMPOSITION

(71) Applicant: SIPCAM INAGRA, S.A., Valencia (ES)

(72) Inventor: Gianluca VALIERI, Valencia (ES)

(21) Appl. No.: 15/313,022

(22) PCT Filed: May 15, 2015
(86) PCT No.: PCT/ES2015/070388

§ 371 Date: Nov. 21, 2016

(30) Foreign Application Priority Data
May 26, 2014 (ES) P201430782

(51) Int. Cl. C11D 1/825; A61K 47/24; A23L 33/10; C05G 3/06; A01N 25/30; A61K 47/10; C11D 1/82

(52) U.S. Cl. CPC C11D 1/825; A61K 47/10; A61K 47/24; C11D 1/82; C05G 3/06; A01N 25/30; A23L 33/10; A23V 2002/00

(57) ABSTRACT

This invention refers to a synergistic composition of surfactants containing glycerine, monopropyleneglycol and organosilicone, to the procedure for preparing it and to the use of this composition as a superwetting agent.

### FIELD OF THE INVENTION

This invention lies within the general field of chemistry and refers in particular to a synergistic surfactant composition.

### STATE OF THE ART

“Wetting agents” are substances that lower the surface tension of water and “superwetting agents” are those substances that reduce surface tension of water below a value of 25 mN/m.

Currently, surfactants are widely used in most industries (cosmetics, agriculture, pharmaceuticals, et.) as described in U.S. Pat. No. 6,653,288 and in patent application CN102440250.

However, despite the large number of compounds that act as surfactants, reducing surface tension below 25 mN/m is very complicated with substances that are not organosilicones.

There is therefore a need to provide a composition that has a “superwetting” effect and that can lower surface tension to below 25 mN/m.

### DESCRIPTION OF THE INVENTION

This invention solves the problems of the state of the art since it provides a composition with which a surface tension can be achieved with values of around 20 mN/m by taking advantage of the synergy of three components.

Thus, in a first aspect this invention refers to a synergistic composition of surfactants (hereinafter, the composition of this invention), featuring glycerine, glycol and organosilicone.

In a preferred embodiment, the glycerine in the composition of this invention is glycerine from the synthesis of bio Diesel.

In a preferred embodiment, the glycol in the composition of this invention is monopropyleneglycol.

In a preferred embodiment, the composition of this invention comprises:
10-90% by weight of glycerine
10-70% by weight of monopropyleneglycol
1-30% by weight of organosilicone.

In another aspect, this invention refers to the use of the composition of this invention as a superwetting agent.

In this invention, superwetting is understood to refer to those substances that lower the surface tension of water below a value of 25 mN/m.

In another aspect, this invention refers to the use of this invention in compositions for insecticides, herbicides, fungicides, food, bio stimulants and fertilisers.

### DESCRIPTION OF THE FIGURES

FIG. 1 shows the surface tension reduction graph of composition B and of composition C. The vertical axis shows the surface tension value of the water solution in mN/m and the horizontal axis shows the concentration value as % p/p of composition B or composition C dissolved in water.

FIG. 2 shows the surface tension reduction graph of composition B and of composition A. The vertical axis shows the surface tension value of the water solution in mN/m and the horizontal axis shows the concentration value as % p/p of composition B or composition A dissolved in water.

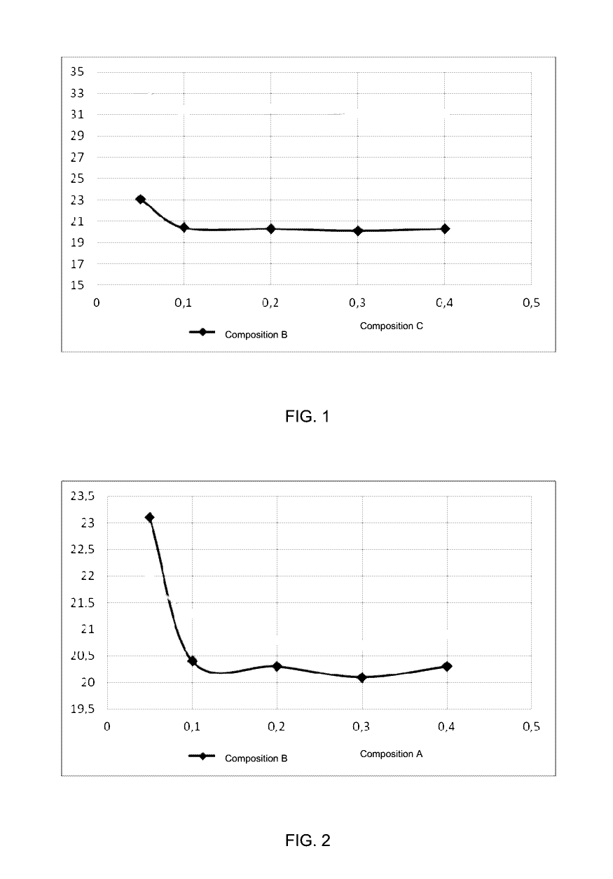

### DETAILED DESCRIPTION OF THE INVENTION

### Example 1: Synergistic Effect of the Compositions of this Invention

To prepare the composition of this invention, unrefined glycerine from the synthesis of bio Diesel was used and was mixed with organosilicone and monopropyleneglycol (MPG). The mixing was produced by simple addition and homogenising of the three components, the preparation of the product being independent of the order in which they are added.

To check the synergistic effect, the surface tension was measured of each individual component, combined two by two and finally the three components of the product together.

The surface tension was measured by preparing the appropriate solutions with mains water and measuring the surface tension value at room temperature (25±2° C.) in a Kruss K9ET plate tensiometer (Wilhelmy platinum plate method). This method is based on measuring the maximum force needed in the vertical direction on a plate in contact with the surface of a liquid in the measuring container so that the liquid separates from the surface. This plate is connected to electronic scales to determine the surface tension value.

The measurement process involves the following steps:
Fill the sample container with the sample to be analysed and place it in the tensiometer equipped with a platinum plate connected to the scales.
Raise the sample container until the surface of the liquid is just below the bottom face of the plate.
Rate the equipment.
Continue to raise the sample container slowly until the plate makes first contact with the surface of the liquid. Stop it as soon as there is a first contact with the liquid.
Turn the gauge wheel until the wheel's indication matches the fixed mark on the equipment. This leaves a reference of the point at which the first contact occurred between the sample and the platinum plate.
Raise the sample 5 mm, submerging the plate and checking that the plate is wetted with the product to be analysed.
Lower the sample container slowly to remove the plate from the product solution until the reference mark aligns with the fixed mark on the equipment again (first contact of the platinum plate with the sample).

The results are shown in the following tables.

TABLE 1
Reduction of surface tension values
at concentrations of 0.025% p/p.

| | | | Surface tension |
|---|---|---|---|
| Glycerine | MPG | Organosilicone | (mN/m) |
| 0.025% p/p | | | 71.7 |
| | 0.025% p/p | | 72.3 |
| | | 0.025% p/p | 29.0 |
| 0.025% p/p | | 0.025% p/p | 28.6 |
| | 0.025% p/p | 0.025% p/p | 28.9 |
| 0.025% p/p | 0.025% p/p | | 68.1 |
| 0.025% p/p | 0.025% p/p | 0.025% p/p | 28.7 |

TABLE 2
Reduction of surface tension values
at concentrations of 0.050% p/p.

| | | | Surface tension |
|---|---|---|---|
| Glycerine | MPG | Organosilicone | (mN/m) |
| 0.050% p/p | | | 68.7 |
| | 0.050% p/p | | 70.3 |
| | | 0.050% p/p | 25.2 |
| 0.050% p/p | | 0.050% p/p | 24.1 |
| | 0.050% p/p | 0.050% p/p | 24.3 |
| 0.050% p/p | 0.050% p/p | | 66.0 |
| 0.050% p/p | 0.050% p/p | 0.050% p/p | 22.5 |

TABLE 3
Reduction of surface tension values
at concentrations of 0.100% p/p.

| | | | Surface tension |
|---|---|---|---|
| Glycerine | MPG | Organosilicone | (mN/m) |
| 0.100% p/p | | | 67.0 |
| | 0.100% p/p | | 69.5 |
| | | 0.100% p/p | 20.9 |
| 0.100% p/p | | 0.100% p/p | 22.2 |
| | 0.100% p/p | 0.100% p/p | 22.9 |
| 0.100% p/p | 0.100% p/p | | 70.6 |
| 0.100% p/p | 0.100% p/p | 0.100% p/p | 20.3 |

As can be seen in Tables 1-3, the effect of the organosilicone prevails over the other individual components at equal concentrations. Even so, an additional reduction of surface tension is achieved to reach 20.3 mN/m, taking advantage of the synergistic effect of the mixture of the three components. Note that it is necessary to use a combination of three components to achieve an additional reduction of surface tension of approximately 10%, compared to organosilicone alone.

To confirm the effect of the reduction of surface tension of composition B and that this effect is maintained in the presence of the three components, two different compositions for the product were tested and the effect of these new formulas (B1 and B2) measured.

TABLE 4
Table for composition of formulas B and variations B1 and B2.

| | Glycerine | Monopropyleneglycol | Organosilicone |
|---|---|---|---|
| Composition | (% p/p) | (% p/p) | (% p/p) |
| B | 56 | 34 | 10 |
| B1 | 53 | 32 | 15 |
| B2 | 58 | 35 | 7 |

Surface tension at 0.2% p/p:

B: 20.3 mN/m
B1: 20.2 mN/m
B2: 20.6 mN/m

At this point it can be seen that composition B is the optimal one, taking into account the compromise between the synergistic effect seen (lowering of surface tension) and the amount of organosilicone to be used.

The composition of this invention (composition B) was then compared with other compositions (composition A and composition C) containing organosilicone mixed with other non-ionic surfactants. In fact, it is known that these substances can improve the effectiveness of organosilicone but from the point of view of reducing the dose.

Table 5 shows that the composition of this invention not only improves the effectiveness in the sense of reducing the dose but also improves it in the sense of achieving a reduction in the surface tension of water, in absolute value terms, attaining a lower surface tension value than that achieved with organosilicone alone or using other potentially synergistic substances in the general sense.

It is thus shown that the new superwetting and synergistic effect is due to the mix of the three compounds of this invention.

TABLE 5
Reduction of surface tension values compared with
other products containing organosilicone.

| | Solution | Solution | Solution |
|---|---|---|---|
| | at 0.1% | at 0.2% | at 0.3% |
| | in water | in water | in water |
| | Surface tension | Surface tension | Surface tension |
| Composition | (mN/m) | (mN/m) | (mN/m) |
| A (repetition 1) | 21.4 | 20.8 | 20.8 |
| A (repetition 2) | 21.3 | 20.9 | 20.7 |
| B (repetition 1) | 20.4 | 20.3 | 20.0 |
| B (repetition 2) | 20.5 | 20.4 | 20.2 |
| C (repetition 1) | 31.4 | 31.7 | 31.0 |
| C (repetition 2) | 31.6 | 31.5 | 31.1 |

Composition A, containing 60% organosilicone, 30% monoethylenglycol and 10% sodium lauryl sulphate.

The composition of this invention, containing 56% glycerine, 34% monopropyleneglycol and 10% organosilicone.

Composition C consisted of 90% canola oil and 10% organosilicone.

As shown by the results in Tables 4 and 5, the composition of this invention (composition B) shows the best reduction of surface tension. Composition B generates lower surface tension values than combination A, although it contains a sixth less of the same organosilicone.

When diluted in water with the same dose (0.2%), composition B generates the same range of water surface tension reduction as composition A. It is important to note that at surface tension levels approaching 20 mN/m, achieving a reduction of 0.5 mN/m is really complicated even though the difference in absolute value may appear small, but it must be remembered that it is not since this is very close to the cancellation of the dosage effect. The same occurs when composition B is compared with composition C.

Extending the range of concentrations comparing composition B and composition A and the same composition B compared to composition C, it can be seen that:
compared with a product containing the same quantity of organosilicone (combination C, 10% organosilicone), the reduction of the surface tension is significantly sharper for the case of this invention (combination B).

TABLE 6
Reduction of surface tension B vs C

| Concentration | | |
|---|---|---|
| in water | | |
| (% p/p) | Composition B | Composition C |
| 0.05 | 23.1 | 33.6 |
| 0.10 | 20.4 | 31.5 |
| 0.20 | 20.3 | 31.6 |
| 0.30 | 20.1 | 31.1 |
| 0.40 | 20.3 | 31.1 |

compared with composition A (with a concentration six times greater of organosilicone) it can be seen that a concentration of 0.1% p/p in water has already exceeded the effect of composition A and maintains this reduction at higher concentrations.

TABLE 7
Reduction of surface tension B vs A

| Concentration | | |
|---|---|---|
| in water | Invention | |
| (% p/p) | (B) | Other A |
| 0.05 | 23.1 | 21.9 |
| 0.10 | 20.4 | 21.3 |
| 0.20 | 20.3 | 20.8 |
| 0.30 | 20.1 | 20.7 |
| 0.40 | 20.3 | 20.9 |

It should be noted that with composition A, water surface tension values below 20.5 mN/m are never generated. On the other hand, all the values generated by composition B are below this value.

1.-6. (canceled)

7. Synergistic composition of surfactants consisting of glycerine, a glycol and organosilicone.

8. Composition of claim 7, which contains:
10-90% by weight of glycerine
10-70% by weight of monopropyleneglycol
1-30% by weight of organosilicone.

9. Use of composition of claim 7 as a superwetting agent.

10. Use according to claim 9 in compositions for insecticides, herbicides, fungicides, food, bio stimulants or fertilisers.

11. Composition of claim 7, wherein the glycerine is glycerine from the synthesis of bio Diesel.

12. Composition of claim 11, which contains:
10-90% by weight of glycerine
10-70% by weight of monopropyleneglycol
1-30% by weight of organosilicone.

13. Use of composition of claim 11 as a superwetting agent.

14. Use according to claim 13 in compositions for insecticides, herbicides, fungicides, food, bio stimulants or fertilisers.

15. Composition of claim 7, wherein the glycol is monopropyleneglycol.

16. Composition of claim 15, which contains:
10-90% by weight of glycerine
10-70% by weight of monopropyleneglycol
1-30% by weight of organosilicone.

17. Use of composition of claim 15 as a superwetting agent.

18. Use according to claim 17 in compositions for insecticides, herbicides, fungicides, food, bio stimulants or fertilisers.
